(19) United States
(12) Patent Application Publication
(10) Pub. No.: US 2004/0018582 A1
(43) Pub. Date: Jan. 29, 2004

(54) Genes and gene products that confer susceptibility to vancomycin derivative antibiotics and methods and assays for identifying bifunctional glycopeptide antibiotics using same

(76) Inventors: Ulrike Eggert, Boston, MA (US); Natividad Ruiz, Princeton, NJ (US); Brian Falcone, Princeton, NJ (US); Arthur Branstrom, East Windsor, NJ (US); Robert Goldman, Rockville, MD (US); Thomas Silhavy, Belle Mead, NJ (US); Daniel Kahne, Princeton, NJ (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/223,130

(22) Filed: Apr. 15, 2003

Related U.S. Application Data
(60) Provisional application No. 60/313,272, filed on Aug. 17, 2001.

(51) Int. Cl. C12Q 1/18

(52) U.S. Cl. 435/32

(57) ABSTRACT

The present invention relates to methods for identifying antibiotic compounds having transglycosylase inhibitory activity or transpeptidation inhibitory activity.

### CROSS-REFERENCE TO RELATED APPLICATIONS

This application claims benefit of provisional application serial No. 60/313,272, filed Aug. 17, 2001, the disclosure of which is hereby incorporated by reference in its entirety.

### FIELD OF THE INVENTION

The present invention relates to methods for identifying compounds having transglycosylase inhibitory activity or transpeptidation inhibitory activity. The compounds are useful as antibiotics.

### BACKGROUND OF THE INVENTION

Vancomycin is the drug of last resort for treating resistant Gram-positive bacterial infections. Emergence of vancomycin resistance presents a serious threat to public health. Vancomycin inhibits the maturation of the peptidoglycan layer surrounding bacterial cells by binding to D-Ala-D-Ala, a dipeptide found in peptidoglycan precursors. J. C. J. Barna et al., Ann. Rev. Microbiol. 38, 339 (1984). Small molecules that affect specific protein functions can be valuable tools for dissecting complex cellular processes. Peptidoglycan synthesis and degradation is a process in bacteria that involves multiple enzymes under strict temporal and spatial regulation. A set of small molecules that inhibit the transglycosylation step of peptidoglycan synthesis were used to discover genes that help to regulate this process. Accordingly, there is a need for a better understanding of the mechanism by which certain antibiotic compounds operate so that steps can be taken to overcome bacterial resistance to antibiotics.

### SUMMARY OF THE INVENTION

The present invention is generally directed to methods of identifying the presence or absence of transglycosylase inhibitory activity in a compound. The method includes the steps of contacting bacterial cells having the wild type yfgL gene with the compound and measuring the antibiotic effectiveness of the compound. The compound is also contacted with a bacterial cells having the mutant yfgL gene. The antibiotic effectiveness is measured. Transglycosylation inhibitory activity is identified by comparing the antibiotic effectiveness of the compound in the cells having the wild type gene with the antibiotic effectiveness in the cells having the mutated gene. If transglycosylation inhibitory activity is present, the compound will have antibiotic effectiveness against the wild type cells and will have decreased antibiotic effectiveness against the mutant cells. If transpeptidation inhibitory activity is present, the compound will have antibiotic effectiveness in both wild type and mutant cells. In certain embodiments, the mutant yfgL gene confers resistance to a transglycosylation inhibitor, such as, for example, teicoplanin or moenomycin.

The compound may be a glycopeptide derivative of vancomycin, teicoplanin, chloroeremomycin or a bifunctional compound comprised of any of the aforementioned compounds or their derivatives linked to another moiety derived from a structurally distinct category of antibiotics that inhibit cell wall synthesis, including, moenomycin, beta lactams, and cephalosporins. In some embodiments, the compound may be a bifunctional glycopeptide derived from an aglycone of vancomycin, such as chlorobiphenyl vancomycin, desleucyl chlorobiphenyl vancomycin. In some embodiments, the compound may be a saccharide derived from moenomycin that is linked to an aglycone by a linker moiety. The first bacterial cell culture may contain mutant and wild type E. coli cells and in some embodiments, the cells are BE 100, BE 101, BE 102 or BE 103.

The present invention is useful for research and for better understanding antibacterial resistance and the regulatory pathways involved in bacterial cell growth and division. In addition, the present invention has clinical applications, such as, for example, for identifying a family of pharmaceutical compounds to be administered for therapeutic purposes.

### DETAILED DESCRIPTION

Resistance to vancomycin arises when microorganisms acquire genes that lead to the substitution of D-Ala-D-Ala by D-Ala-D-Lac T, to which vancomycin does not bind. D. Bugg et al., Biochemistry 30, 2017 (1991). Remarkably, vancomycin derivatives with a hydrophobic substituent on the carbohydrate moiety are effective against vancomycin resistant strains even though they contain the same peptide-binding pocket as vancomycin. R. Nagarajan et al, J. Antibiot. 42, 63 (1989). These derivatives may have a different mechanism of action than vancomycin. Other explanations have been proposed. D. H. Williams et al., Angew. Chem. Int. Ed. 38, 1173 (1999). Unlike vancomycin, they retain activity against both vancomycin sensitive and resistant strains even when the peptide-binding pocket is damaged. In vivo, they block a different step of peptidoglycan synthesis than vancomycin. M. Ge et al, Science 284, 507 (1999). In addition, they kill bacteria rapidly whereas vancomycin only stops growth. S. A. Zelenitsky et al., Antimicrob. Agents Chemother. 41, 1407 (1997).

Because the vancomycin derivatives affect cells differently than vancomycin, i.e. by producing a different phenotype, it might be possible to identify genes involved in the cellular response to these compounds using a chemical genetics approach. The synthesis of peptidoglycan from its disaccharide precursor involves numerous enzymes with overlapping functions that are subject to tight temporal and spatial regulation. J.-V. Holtje, Microbiol. Mol. Biol Rev. 62, 181 (1998). Most of the major enzymes in peptidoglycan synthesis, the transglycosylases and transpeptidases, have been identified, but how these enzymes are regulated remains poorly understood. Vancomycin blocks the transpeptidation step of peptidoglycan synthesis and kills cells slowly. Glycolipid derivatives of vancomycin, however, block the transglycosylation step of peptidoglycan synthesis and provoke a rapid lethal response in cells. Moenomycin, another transglycosylase inhibitor, also induces a rapid lethal response in cells. Y. v. Heijenoort et al., J. Gen. Microbiol. 1333, 667 (1987). Thus, inhibiting the transglycosylation step of peptidoglycan synthesis may activate a pathway that triggers rapid cell death. The components of this pathway may be identified by selecting for mutants that are resistant to small molecules that inhibit transglycosylation. One gene responsible for susceptibility of Escherichia coli cells to killing by glycolipid derivatives of vancomycin was identified, establishing a genetic basis for differences in activity between these compounds and vancomycin.

Mutants resistant to three different transglycosylase inhibitors, chlorobiphenyl vancomycin, desleucyl chlorobiphenyl vancomycin and moenomycin were identified. Vancomycin and chlorobiphenyl vancomycin have the following structures:

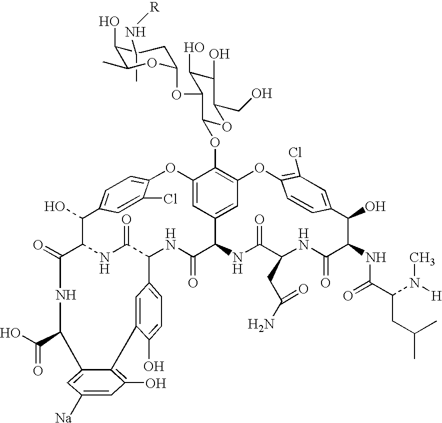

Vancomycin: R═H Chlorobiphenyl Vancomycin:

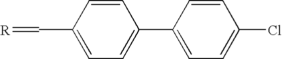

Desleucyl chlorobiphenyl vancomycin has the following formula:

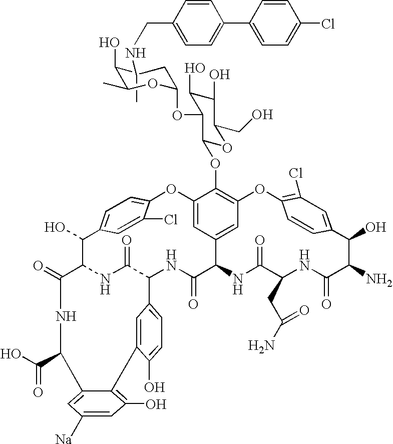

Moenomycin has the following formula:

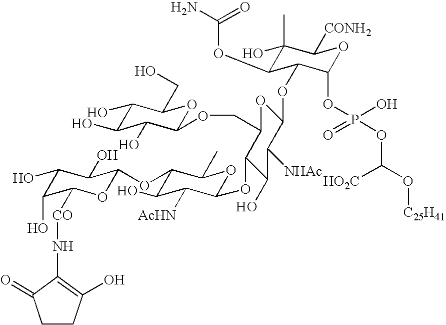

Moenomycin binds directly to certain bacterial transglycosylases. W. Vollmer et al., J. Biol. Chem. 274, 6726 (1999). Chlorobiphenyl vancomycin inhibits transglycosylation by binding to the D-Ala-D-Ala terminus of the peptidoglycan precursor lipid II and also by binding to components of the transglycosylation complex. M. Ge et al, Science 284, 507 (1999). Desleucyl chlorobiphenyl vancomycin cannot bind D-Ala-D-Ala and is proposed to inhibit transglycosylation primarily by the latter mechanism. M. Ge et al, Science 284, 507 (1999).

The present inventions are directed to methods of identifying a compound having transglycosylase inhibitory activity. The method includes contacting the compound with cells having the wild type yfgL gene and then measuring the antibiotic effectiveness and contacting the compound with cells having the mutant yfgL gene and measuring the antibiotic effectiveness the compound. Transglycosylation inhibitory activity is identified by the compound having antibiotic effectiveness in the wild type cells and having decreased antibiotic effectiveness in the mutant cells. For example, using this assay, teicoplanin was found to block the transglycosylation step of peptidoglycan synthesis.

The bacterial cell cultures may contain cells that are mutant and wild type E. coli cells. The mutant cells can be, for example, BE 100, BE 101, BE 102 and BE 103 cells. The mutant and wild type cells may also be homologues of the yfgL gene. The yfgL gene upregulates lytic transglycosylases. In certain embodiments, the mutant are resistant to more than one compound.

“Transglycosylase inhibitory activity” as referred to herein means an ability to inhibit the polymerization of the disaccharide Lipid II to form the carbohydrate chains of peptidoglycan, a reaction catalyzed by enzymes having transglycosylase activity. In some embodiments polymerization is inhibited 25, 50, 75, 80, 85, 90, 95, 99 or 100%.

“BE 100, BE 101, BE 102 and BE 103” as referred to herein means isolates of BAS849 (MC4100 ΔlamB106 imp 4213).

“Functional homologues” as referred to herein means other genes involved in the regulation of transglycosylation. “Sequence homologues” as referred to herein means statistically significant similarities in sequence to yfgL.

### EXAMPLES

In order that the invention disclosed herein may be more efficiently understood, examples are provided below. It should be understood that these examples are for illustrative purposes only and are not to be construed as limiting the invention in any manner.

Mutants resistant to transglycosylase inhibitors were obtained by growing E. coli imp on plates impregnated with chlorobiphenyl vancomycin, desleucyl chlorobiphenyl vancomycin or moenomycin. Vancomycin and its analogs do not penetrate the outer membrane of most E. coli strains. An E. coli imp strain was used as a test strain organism for selecting mutants because E. coli is the best understood bacterial species. E. coli membrane preparations have been used for many of the mechanistic studies on vancomycin and its derivatives. The imp mutation alters the permeability of the outer membrane and confers sensitivity to vancomycin and its carbohydrate derivatives as well as to moenomycin. BE100 is an isolate of BAS849 (MC4100 ΔlamB106 imp 4213). B. A. Sampson et al., Genetics 122, 491(1989). Three mutants, BE101, BE102 and BE103, resistant to each of these antibiotics were isolated (see Table 1 below).

TABLE 1

| | Wild-type | Mutant | Mutant | Mutant |
|---|---|---|---|---|
| Antibiotics/Strain | (BE100) | BE101 | BE102 | BE103 |
| Vancomycin | 0.8 | 1.6 | 1.6 | 1.6 |
| Chlorobiphenyl | 0.2 | 3.2 | 1.6 | 3.2 |
| Vancomycin | | | | |
| Desleucyl | 25 | >125 | >125 | >125 |
| Chlorobiphenyl | | | | |
| Vancomycin | | | | |
| Moenomycin | 0.03 | 1.6 | 0.8 | 3.2 |
| Teicoplanin | 0.25 | 16 | 3 | 4 |
| Penicillin G | 3.2 | 3.2 | 1.6 | 3.2 |

Table I presents minimum inhibitory concentrations (MICs) (μg/ml) for wild-type and mutant strains. MICs were determined against strains grown in LB medium in a standard microdilution format. The MIC is defined as the lowest antibiotic concentration that resulted in no visible growth after incubation at 35° C. for 22 hours. For desleucyl chlorobiphenyl vancomycin higher numbers could not be obtained due to lack of solubility. Mutant BE101 was raised on chlorobiphenyl vancomycin, BE102 was raised on desleucyl chlorobiphenyl vancomycin and BE103 on moenomycin.

The mutations do not cause a significant change in growth rates. Some antibiotics, for example the β-lactams, show a decrease in their bactericidal effectiveness against slow-growing cells. E. Tuomanen et al., J. Gen. Microbiol. 132, 1297 (1986). The rate of killing is independent of the growth rate. Each mutant was resistant not only to the transglycosylase inhibitor on which it was raised, but also to both of the other antibiotics. For example, moenomycin was approximately 27-100 times less active in all three of the resistant strains compared to the wild-type strain, whereas chlorobiphenyl vancomycin was approximately ten times less active. The mutants were also resistant to the glycopeptide antibiotic teicoplanin, another inhibitor of the transglycosylation step of peptidoglycan synthesis. R. Kerns et al., J. Am. Chem. Soc. 122, 12608 (2000). However, the mutants did not show any resistance to vancomycin.

The mutants remained as sensitive as the parent strain BE 100 to antibiotics that inhibit peptidoglycan synthesis but do not block transglycosylation e.g., transpeptidase inhibitors. Examples of transpeptidase inhibitors include, but are not limited to, ampicillin, penicillin or cefoxitin (p-lactams). Compounds that inhibit cytoplasmic steps of peptidoglycan synthesis include, but are not limited to, fosfomycin and cycloserine. The mutants were also sensitive to antibiotics that target other essential cellular functions, including erythromycin (a macrolide) and kanamycin or gentamicin (aminoglycosides). The fact that the mutants demonstrate increased minimum inhibitory concentrations (MICs) to molecules that block the transglycosylation step of peptidoglycan synthesis, but not to antibiotics with other modes of action, rules out non-specific mechanisms of resistance such as altered membrane permeability. Inhibition of transglycosylation in sensitive cells may activate a common pathway that leads to rapid cell death. This pathway might be blocked in the mutants, resulting in resistance to rapid cell death upon treatment with transgiycosylase inhibitors, even at the increased MICs.

Set forth below is Scheme 1, which demonstrates the final stage of peptidglycan biosyntheis. Transglycosylase enzymes polymerize lipid II into long polysaccharide chains of immature peptidoglycan that are then cross-linked by transpeptidases. Chlorobiphenyl vancomycin, moenomycin and teicoplannin are examples of compounds that inhibit the transglycosylation step. Examples of compounds that inhibit the transpeptidation step include, but are not limited to vancomycin and β-lactams.

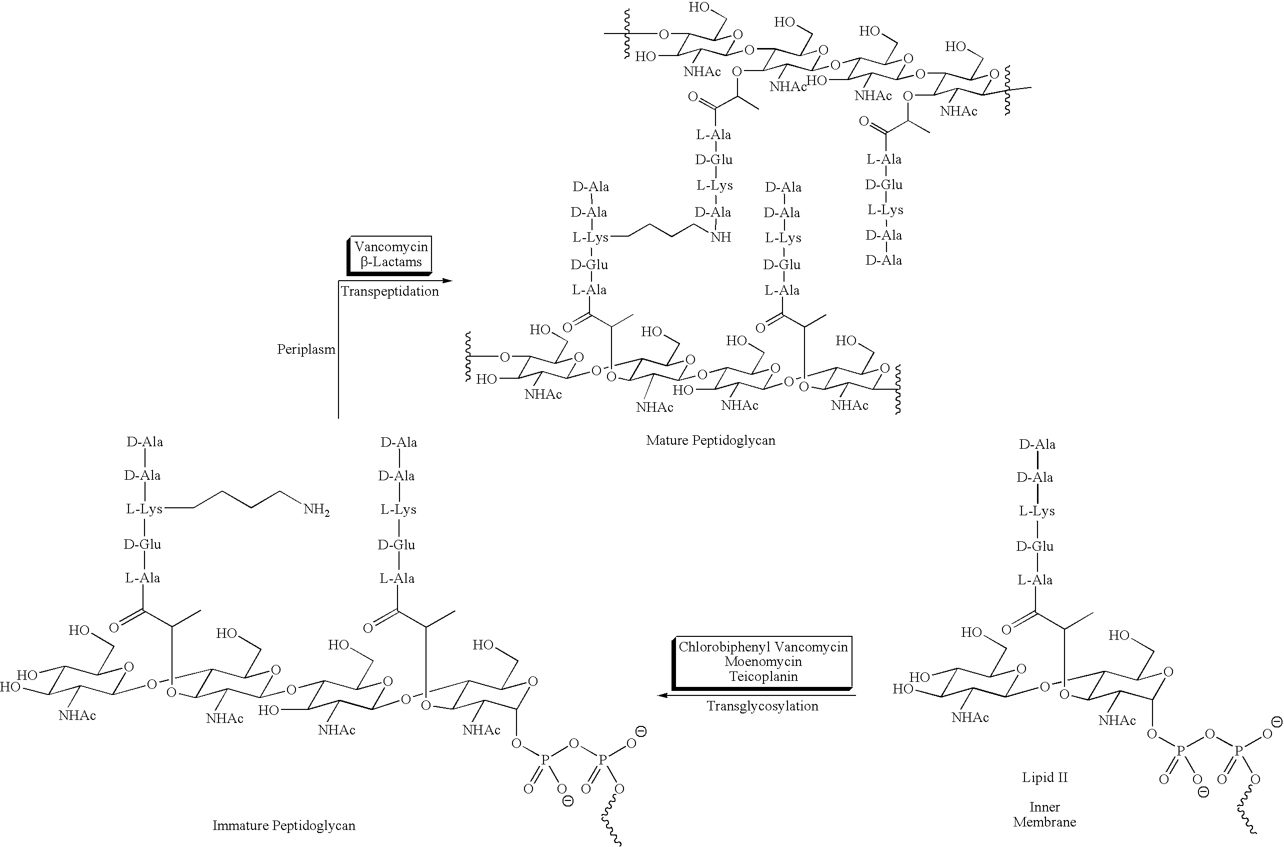

The survival rates were measured of resistant and sensitive bacteria when exposed to vancomycin and the three transglycosylase inhibitors discussed above (i.e., chlorobiphenyl vancomycin, desleucyl chlorobiphenyl vancomycin and moenomycin). Vancomycin at its MIC prevented growth of all the strains but did not kill them. The results are presented graphically in FIGS. 1C and 1D below.

[image]

FIGS. 1C and 1D represent time-kill curves of wild-type BE100 (C) and mutant strain BE101 (D) in the presence of antibiotics at their MICs. A standard microdilution assay was performed in LB. Samples were removed and serial dilutions were plated onto antibiotic-free agar after 15, 30, 60, 120 and 240 minutes of exposure to each antibiotic. Drug concentrations were 0.8 μg/ml and 1.6 μg/ml for vancomycin (♦) and 0.3 μg/ml and 3.2 μg /ml for chlorobiphenyl vancomycin (●) for wild-type BE100 and mutant BE101, respectively.

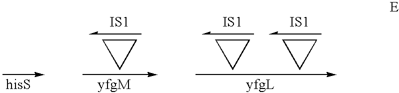

FIG. E represents a genetic map of the hisS operon. The mutant strains BE101, BE102 and BE103 were obtained from parent strain BE100 by plating onto LB agar containing five times the MIC for a given antibiotic. Individual colonies were purified. Genetic mapping and DNA sequence analysis revealed insertion mutations in yfgM and yfgL. The insertions are IS1E elements located at base 342 inyfgM (BE101) and at bases 171 (BE102) and 1024 (BE103) in yfgL (where +1 refers to the A of their predicted ATG start codons). Arrows indicate direction of transcription.

Chlorobiphenyl vancomycin caused the number of colony-forming units (CFUs) of the wild-type strain to decrease by four orders of magnitude in two hours (see FIG. 1C). However, it did not reduce significantly the CFUs of the mutant strain in two hours even at a concentration ten times higher than that used against the wild-type strain (see FIG. 1D). The other transglycosylase inhibitors also showed a significant decrease in their ability to kill mutant strains. The bacteriostatic activity of the transglycosylase inhibitors against the mutant strains is consistent with inactivation of a gene(s) that would otherwise activate a bactericidal response. The mutation that causes resistance might be a recessive null mutation.

In order to identify the mutated gene(s) that lead to resistance, standard genetic methods were used to map the mutations in all three strains to minute 56.8 in the E. coli genome. Strains containing Tn10 insertions in nearby genes purC and yfhS were obtained from the collection of Singer et al. M. Singer et al., Microbiol. Rev. 53, 1 (1989). Strains containing kanamycin resistance cassettes in pbpC and yfgJ were constructed using the methods described by D. Yu et al., Proc. Natl. Acad. Sci. U. S. A. 97, 5978 (2000). Linkage between these markers and the mutation conferring resistance to the transglycosylase inhibitors was determined by generalized transduction using P1vir. T. J. Silhavy et al., Experiments with Gene Fusions (Cold Spring Harbor Laboratory Press, Cold Spring Harbor, N.Y., 1984). Sequencing of this region showed that IS1E elements had inserted into either yfgL or yfgM in the hisS operon in all three mutants (FIG. 1E). M. Umeda et al., Gene 98, 1 (1991).

To verify that the mutations in yfgL or yfgM were responsible for the resistant phenotype, complementation experiments were performed. The chromosomal region encoding the wild-type hisS, yfgM and yfgL genes (with their promoter region) was inserted into a plasmid and introduced into both the wild-type and mutant strains. hisS, yfgM, yfgL and the region 100 base pairs upstream of hisS were PCR amplified from the chromosomes of BE100 and BE103 by using the primers yfgM-N1 (5′-AAA GAA TTC CGT GTA TGA TTG AAC CCG C-3′) (SEQ ID NO:1) and yfgLC-1(5′-TAC ACC GTC TTC TGT GCC A-3′) (SEQ ID NO:2), purified, digested with EcoRI and Kpnl and cloned into the multicloning site of pUC19. To reduce copy number, an EcoRI to HindIII fragment was cloned from this pUC19 derivative into the EcoRI to HindIII sites of pBR322. yfgL alone was PCR amplified from the chromosome of BE100 using the primers LEco-N (5′-AAA GAA TTC GAG AGG GAC CCG ATG CAA-3′) (SEQ ID NO:3) and LHin-C (5′-AAA AAG CTT GAT TAA CGT GTA ATA GAG TAC A-3′) (SEQ ID NO:4), digested with HindIII and EcoRI and cloned into the multicloning site of pBAD18. recA::kan was moved into BE100 and the mutants by transduction.

Plasmids containing either the intact or mutated hisS operon or yfgL alone were transformed into the recombination-deficient strains and transformants were selected on LB plates with 5 μg/ml ampicillin. T. J. Silhavy et al., Experiments with Gene Fusions (Cold Spring Harbor Laboratory Press, Cold Spring Harbor, N.Y., 1984). The strains containing the pBAD vector were induced with 0.1% arabinose. The transformants were grown in liquid LB with 5 μg/ml ampicillin and their resistance phenotypes determined by MIC. The mutant strains carrying the wild-type genes in trans regained sensitivity to transglycosylase inhibitors, as determined by MIC and showed a similar bactericidal response as the wild-type strain. The wild-type strains carrying either the mutant or the wild-type genes in trans retained the wild-type phenotype. Thus, the loss of a functioning copy of yfgM or yfgL (or both) in the mutant strains leads to the resistant phenotype.

The yfgL gene product may be involved in the bactericidal response to compounds that inhibit transglycosylation. However, because an IS insertion in yfgM prevents expression of yfgL by polarity, the role of yfgM remained ambiguous. To determine whether yfgM plays any role in cell death upon transglycosylase inhibition, yfgL and yfgM were cloned separately into an inducible vector and introduced it into the mutant strains. The mutants became sensitive to transglycosylase inhibitors only if yfgL production was induced. In the absence of inducer, or when yfgM alone was expressed, the mutant strains remained resistant. Thus, the bactericidal effects of transglycosylase inhibition require a functional yfgL gene.

Sequence analysis of the gene products suggests that yfgL is a lipoprotein located on the inner surface of the outer membrane of the bacterial cell. K. Yamaguchi et al., Cell 53, 423-432 (1988). One possible mechanism by which yfgL causes cell death in the presence of transglycosylase inhibitors is that it affects the regulation of lytic transglycosylases (autolysins). It is known that the bactericidal action of antibiotics in Streptococcus pneumoniae is related to the autolysin LytA. A. Tomasz et al., Nature 227, 138 (1970). It has been suggested that a two-component signaling system controls death in this organism. R. Novak et al., Nature 399, 590 (1999). Other lysis pathways are also possible. T. G. Bernhardt et al., Science 292, 2263 (2001). These enzymes break peptidoglycan bonds to permit the incorporation of new peptidoglycan during growth and cell division. J.-V. Holtje, Microbiol. Mol. Biol Rev. 62, 181 (1998). Some lytic transglycosylases are located on the inner surface of the outer membrane and could cause rapid cell death if they are inappropriately activated. YfgL most likely is not a lytic transglycosylase because it has no homology to any known lytic transglycosylase. J. Lommatzsch et al., J. Bacteriol. 179, 5465 (1997). Bacterial strains that do not produce the wild-type yfgL gene product make considerably more peptidoglycan than the parent strains, which is consistent with the hypothesis that the normal function of yfgL is to up-regulate lytic transglycosylases. Incorporation of ¹⁴C-labeled UDP-GlcNAc into the peptidoglycan in membrane preparations of BE100 and the mutants was measured as described in A. A. Branstrom et al., FEMS Microbiology Lett. 191, 187 (2000). The mutant strains were able to incorporate approximately 18% of the radiolabeled UDP-GlcNAc, whereas the wild-type strain incorporated only 5%. This could be due to increased activity of the transglycosylase enzymes or failure of the lytic transglycosylases to degrade peptidoglycan.

Sequence homologues of yfgL can be identified in many Gram-negative organisms, but not in Gram-positive bacterial species, possibly due to the fact that the latter do not have an outer membrane. Enterococcal strains, which are Gram-positive, behave similarly to permeable E. coli in that they die very rapidly upon exposure to glycolipid derivatives of vancomycin. S. A. Zelenitsky et al., Antimicrob. Agents Chemother. 41, 1407 (1997). The bactericidal behavior suggests that functional homologues of yfgL exist in enterococcal strains even though homologous proteins have not been identified. Thus, transglycosylases are particularly good targets for the design of new antibiotics because they are extracytoplasmic and because their inhibition provokes a bactericidal response.

The discovery that yfgL confers resistance to glycolipid derivatives of vancomycin but not to vancomycin itself establishes a genetic basis for the activity differences between these compounds. The yfgL gene gives rise to a discernable phenotype in the presence of small molecules that perturb the transglycosylation step of peptidoglycan synthesis. It is thought that yfgL is involved in regulating peptidoglycan synthesis at some level. Small molecules have been used previously to identify regulatory networks. P. J. Alaimo et al., Curr. Op. Chem. Biol. 5, 360 (2001). The classic example involves the elucidation of the cyclophilin/calcineurin network in mammalian cells using cyclosporin. J. Liu et al., Cell 66, 807 (1991); S. L. Schreiber, Science 251, 283 (1991). Microbial regulatory networks are simpler to study using a chemical genetics approach because it is easier to select for mutants and analyze them genetically. Small molecule probes of various kinds should be useful for identifying other “non-essential” genes that regulate essential enzymes in bacteria. In the meantime, understanding how yfgL and its putative counterparts in other organisms trigger cell death upon exposure to transglycosylase inhibitors may lead to a better understanding of the regulatory networks involved in bacterial cell growth and division.

Each of the foregoing references is incorporated herein by reference in its entirety. Various modifications of the invention, in addition to those described herein, will be apparent to those skilled in the art. Such modifications are intended to fall within the scope of the appended claims.

What is claimed is:

1. A method of identifying the presence or absence of transglycosylase inhibitory activity in a compound, comprising:
contacting bacterial cells having the wild type yfgL gene with said compound;
measuring the antibiotic effectiveness of said compound in said cells,
contacting bacterial cells having a mutated yfgL gene with said compound; and
measuring the antibiotic effectiveness of said compound in the cells having the mutated gene.

2. The method of claim 1 further comprising the step of comparing the antibiotic effectiveness of said compound in said cells having the wild type gene with the antibiotic effectiveness of said cells having the mutated gene,
wherein transglycosylation inhibitory activity is identified by said compound having antibiotic effectiveness in said cells having the wild type gene and having decreased antibiotic effectiveness in said cells having the mutated gene.

3. The method of claim 1 wherein said cells are wild type E. coli cells.

4. The method of claim 3 wherein said wild type E. coli cells are BE 100, BE 101, BE 102 or BE 103.

5. The method of claim 1 wherein said cells are mutant E. coli cells.

6. The method of claim 5 wherein said mutant E. coli cells are BE 100, BE 101, BE 102 or BE 103.

7. The method of claim 1 wherein said mutant yfgL gene confers resistance to a transglycosylation inhibitor.

8. The method of claim 1 wherein said compound is teicoplanin or moenomycin.

9. The method of claim 1 further comprising the step of comparing the antibiotic effectiveness of said compound in said cells having the wild type gene with the antibiotic effectiveness of said cells having the mutated gene,
wherein transpeptidation inhibitory activity is identified by said compound having antibiotic effectiveness in said cells having the wild type gene as well as in said cells having the mutated gene.

10. A method of identifying the presence or absence of transglycosylase inhibitory activity in a compound, comprising:
contacting bacterial cells having a sequence or functional homologue of the wild type yfgL gene with said compound;
measuring the antibiotic effectiveness of said compound in said cells,
contacting bacterial cells having a mutated sequence or functional homologue of the yfgL gene with said compound; and
measuring the antibiotic effectiveness of said compound in said cells having a mutated sequence or functional homologue of the yfgL gene.

11. The method of claim 10 wherein said cells are wild type E. coli cells.

12. The method of claim 11 wherein said wild type E. coli cells are BE 100, BE 101, BE 102 or BE 103.

13. The method of claim 10 wherein said cells are mutant E. coli cells.

14. The method of claim 13 wherein said mutant E. coli cells are BE 100, BE 101, BE 102 or BE 103.

15. The method of claim 10 wherein said mutation confers resistance to transglycosylation inhibitors.

16. The method of claim 10 wherein said compound is teicoplanin or moenomycin.

17. The method of claim 10 further comprising the step of comparing the antibiotic effectiveness of said compound in said cells having the wild type gene with the antibiotic effectiveness of said cells having the mutated gene,
wherein transglycosylation inhibitory activity is identified by said compound having antibiotic effectiveness in said cells having the wild type gene and having decreased antibiotic effectiveness in said cells having the mutated gene.

18. The method of claim 10 further comprising the step of comparing the antibiotic effectiveness of said compound in said cells having the wild type gene with the antibiotic effectiveness of said cells having the mutated gene,
wherein transpeptidation inhibitory activity is identified by said compound having antibiotic effectiveness in said cells having the wild type gene as well as in said cells having the mutated gene.
